(19) United States
(12) Patent Application Publication
(10) Pub. No.: US 2003/0212105 A1
(43) Pub. Date: Nov. 13, 2003

(54) Novel anti-fertility agent

(76) Inventors: Shyam Sunder Agrawal, New Delhi (IN); Surendra Bodakhe, New Delhi (IN); Roma Dewan, New Delhi (IN); Pooja Dhameja, New Delhi (IN)

Correspondence Address: FOLEY AND LARDNER, SUITE 500, 3000 K STREET NW, WASHINGTON, DC, 20007, US

(21) Appl. No.: 10/203,096

(22) PCT Filed: Dec. 7, 2000
(86) PCT No.: PCT/IN00/00122

(51) Int. Cl. A61K 31/445

(52) U.S. Cl. 514/317

(57) ABSTRACT

The invention identifies a novel anti-fertility agent, roxatidine, useful for inhibiting or controlling the fertility of female mammals, without side effects.

### TECHNICAL FIELD

The present invention relates a method of controlling fertility using roxatidine as an anti-fertility agent.

### BACKGROUND ART

Histamine or beta amino ethyl imidazole was synthesized for chemical curiosity before its biological significance was known. It was detected as a uterine stimulant in extracts of ergot. Histamine shows its action by acting on histaminergic receptors. Ash & Schild (1966) classified the receptors into H₁ and H₂ receptors. Sir James Black (1972) developed the first H₂ blocker and confirmed the classification of the receptors. It was reported that histamine might provide the chemical stimulus for fixation of placenta [Wislocki et al (1938)]. H₂ receptors are predominantly present in the uterus. It was demonstrated that topical application of Diphenhydramine HCl inhibited the development of decidouma [Shelesnyak, (1952)].

McNeil & Verma (1975) confirmed the presence of H₂-receptors in rat uterus and found that the stimulation of these receptors leads to release of catecholamines in rat uterus, which is ultimately responsible for causing relaxation. Thus, H₂ blockers may lead to contraction of the rat uterus or prevent its relaxation.

The role of histamine in the context of controlling fertility can be better understood with the principles behind fertilization. After the fertilized ovum enters the uterine cavity, it transforms into blastocysts and attempts to implant itself into the endometrium. The hormones secreted during pregnancy are Estrogen and Progesterone HCG. The level of both hormones increases with the progression of pregnancy, reaching the peak at term. Decidualization or the transformation of uterine stromal cells to multi—nucleate cells with abundant cystoplasmic filaments, occurs in a sensitized endometrium, during a limited period (about 25^{th} day of menstrual cycle in human beings & day 4 in rats). Specific alteration in nucleic and protein synthesis is believed to be associated with the process, and it is a progestrone dependent event.

Uterine ability to undergo decidualization is determined by a sequence of events which begins with the action of estrogen, causing division of stem cells, the progeny providing precursor cells for decidualization, which becomes competent predecidual cells in synthesizing DNA in response to a phase of estrogen action during progestation, but without undergoing division. The stimulus for differentiation into primary decidual cells is mediated by histamine, released by interaction of the blastocyst with uterine elements, or provided by experimental stimulation. The predecidual cell has a temporary potential for transformation and if not stimulated, degenerates within a day or two.

The estrogen secreted by the ovary, stimulates release of histamine that acts on hormonally prepared endometrium to induce decidualization and preparation of decidual tissues into which the blastocyst can embed. (M. C. Shelesnyak, 1959).

Nidation is a sequence of overlapping phases, each characterized by predominating events. The first phase is Estrogen priming which includes proestrus and estrus, and is characterized by estrogen dominance. In this stage there is stimulation of turnover of uterine components and formation of the primary endometrium, thus, conferring upon the uterus the potential to respond to decidual induction. Priming, gives way to the second phase, which is called sensitization. This phase is characterized by coitally augmented infiltration of the uterus by leucocytes and the concomitant accumulation of histamine. This phase is completed by the estrogen surge which produces the phase of sensitization, and thus the uterine sensitivity is established.

At this stage the blastocyst enters, sheds its Zona-pellucida and induces histamine release which initiates decidualization. Once decidualization is underway, the blastocyst begins penetration of the uterine epithelium and embeds itself in the nidus. Proliferation of trophoblastic and embryonic tissues begin with implantation and nidation is complete.

Thus, there is a strong suggestion that histamine plays a positive role in different stages of reproduction, i.e. ovulation, implantation and pregnancy.

Brandon & Wallis (1977) reported that treatment with a combination of Mepyramine, a H₁ antagonist and Burimamide, a H₂ receptor antagonist during early pregnancy in rats resulted in a fall in the number of sites of blastocyst attachment. Thus, histamine has been shown to play a definite role in the implantation of the blastocysts. Based on experiments in rats wherein H₂-receptor blocker inhibited decidualisation induced by histamine, it was observed that histamine may be the nonspecific chemical agent involved in initiating the nidatory response in the uterus. Thus, attempts to control or block histamine may provide anti-fertility agents. Anti-fertility agents can broadly be divided into two categories, namely, those that prevent ovulation and act before fertilization, popularly called “contraceptives” and agents that act after fertilization, called “interceptives”.

In case of H₂ blockers with significant anti-fertility activity, the post-implantation loss is greater than pre-implantation loss, this shows that they act by bringing about resorption of implants [Agarwal & Arvinda, (1992)].

Potent H₂ antagonists are imidazoles of the N^{T}H type, a form required for maximal H₂ antagonistic activity. Burimamide (N-[4-(1H imidazolyl-5-yl) butyl]-N′methylthiourea being an imidazole derivative, has an affinity for H₂ receptors.

Substitution of one methylene group in the side chain of burimamide with an isosteric sulphur atom and addition of one methyl group at position 5 on the imidazole ring results in another active compound, metiamide, the dominating neutral tautomer in the nth form. Metiamide is not marketed, because of its side effects which include agranulocytosis attributed to the thiourea function.

Interestingly, the cyanoguanidine function is iso-electronic with the thiourea group. This substitution furnishes cimetidine which is devoid of undesirable side effects. The N^{T}H tautomer of cemitidine also predominates and this drug competitively inhibits the action of histamine at H₂ receptors. It was first marketed in the year 1976. Cimetidine, Ranitidine and such derivatives are known H₂-receptor antagonists. These drugs have other widespread applications such as inhibition of gastric acid secretion, etc.

When the methyl imidazolyl ring of cemitidine is replaced by a dimethyl amino methyl furyl ring, and the cyanoguanidine functionality is replaced by an isosteric methyl nitro ethenediamine moiety and the resultant compound is Ranitidine which is 4 to 10 times more potent than cemitidine.

Famotidine is a H₂ antagonist in which the N-Methyl-2-nitro-1,1-ethene-diamine group of nizatidine is replaced by the amino sulphonyl imidamine function and the basic dimethyl amino ethyl group is replaced by the guanidine functionality.

On the basis of stereo structure inhibitory activity relationships of a series of analogs, it has been shown that the molecular conformation of neutral antagonists revealed by X-ray crystal analysis reflects a folded form suitable for binding to the receptor, although the crystal structure of the cationic molecule such as the hydrochloride salt always shows the non-extended conformation. The large conformational change caused by the acid appears to be a common feature of the antagonists cemitidine, ranatidine & famotidine, all of which have three fundamental structures.

1. the substituted heteroaromatic ring;

2. the connection via a methyl thioethyl chain to an end group; and

3. an essentially neutral urea like end group.

Based on the fact that histamine antagonists are capable of acting as anti-fertility agents, the applicants undertook a study to screen imidazole derivatives for their possible anti-fertility activity. The applicants found to their surprise that certain imidazole derivatives which act as H₂ blockers exhibited substantial anti-fertility activity.

The effect of these drugs on fertility has been studied and documented by a few workers. For example, in a study by S. S. Agrawal and S. Aravinda (Indian Journal of Pharmacology 1995; 27: 40-42), it was found that oral administration of H₂-receptor blockers, cimetidine and ranitidine, to pregnant female rats daily for 1-7 days post coitum exhibited anti-implantation effects. These antagonists did not possess oestrogenic/anti-oestrogenic activity. It appears that these compounds probably cause an inhibition of the local effect of histamine in the uterus on implantation of the fertilised ovum which may be mediated via the predominant uterine H₂-receptors.

Similarly, in a study by Ahmed, Mohamed A. [Egypt, J. Pharm. Sci., 37(1-6), 539-551 (1996)], the possible effect of chronic administration of histaminergic H₂-receptor antagonist (ranitidine) on female fertility was studied. In this study, Ranitidine was administered (either 75 mg/kg i.v. or 150 mg/kg orally) daily to adult female albino rats for 8 weeks. Chronic administration of ranitidine either orally or i.v. could exhibit a significant inhibitory effect on female fertility with subsequent inhibition of ovulation as well as a significant drop in the serum concentration levels of both estradiol and progesterone. The inhibitory effect of ranitidine was reversed completely after injection of pregnant male serum (PMS) gonadotrophin in a dose of 10 lu for 7 days and after stopping the administration of the drug for 8 weeks (after 8 weeks of daily i.v. or oral administration). However, in this study, ranitidine was administered prior to the fertilization of the ovary. The use in this case of ranitidine is as a “contraceptive” than “interceptive”. The effects of an anti-fertility agent when administered prior to fertilization are bound to be different as it has different biochemical reactions as compared to administration after fertilization.

Roxatidine is a new type of H₂ receptor antagonist differing from cemitidine, ranitidine and famotidine in stricture and activity. Roxatidine is not a competitive inhibitor of the H₂ receptor. One reason could be that the effect of the hydrochloride salt on the molecular confirmation is not significant and both the free and cationic molecules have similar extended forms. It is also likely that the binding site for roxatidine on the H₂ receptor is different from that of the other antagonists. However, side effects are absent when administered. Although several studies have been conducted in the past on the use of imidazole derivatives as anti-fertility agents, there has been no study on roxatidine. Also, there is no report of roxatidine as a H₂-receptor blocker. Since the structure and behaviour of roxatidine is different from related imidazole drugs, the results of studies in the past cannot be extrapolated to roxatidine. Further, the structure and activity of roxatidine is much different as compared to ranitidine. Also, the method of administration of roxatidine and ranitidine are different. After much research, the applicants have identified the role and the use of roxatidine in controlling and/or preventing fertilization. The applicants observed unexpected and surprising results during their study on roxatidine in the control of fertilization in mammals.

### OBJECTS OF THE INVENTION

The main object of the invention is to provide a novel anti-fertility agent.

Yet another object is to provide a method for the control of fertility employing roxatidine as an anti-fertility agent.

### SUMMARY OF THE INVENTION

Accordingly, the invention identifies a novel anti-fertility agent, roxatidine, useful for inhibiting or controlling the fertility of female mammals, without side effects.

### DETAILED DESCRIPTION OF THE INVENTION

In accordance with the above and other objectives, the invention provides a novel anti-fertility agent roxatidine. This compound has been found to act as a H₂-receptor antagonist and exhibits anti-fertility activity. The anti-fertility activity exhibited by roxatidine is primarily due to its ability to block H₂ receptors in the uterus since histamine is known to play a pivotal role in nidation, ovulation and implantation.

When the anti-fertility agent of the invention was tested in animal models, it was observed that the action of histamine on its receptors in implantation has been inhibited. As is known, there is a release of esterogen from the ovary prior to implantation of the blastocyst and this is followed by liberation of histamine from the uterus. This histamine is considered to be responsible for deciduoma formation. It is also an accepted fact that deciduoma formation is essential for normal pregnancy. Interference with histaminic produced deciduoma. The present study conducted by the applicants confirm the inhibition of decidual cell reaction. The applicants surprisingly found that roxatidine when administered in animal models is effective in inhibiting implantation and therefore, it could be used as a potential anti-fertility agent.

Accordingly, the invention provides a method for controlling or preventing fertility in mammals, said method comprising the step of administering a therapeutically effective amount of roxatidine to the subject. In other words, the invention provides a method whereby abortion is induced in the subject (mammal) or administration of roxatadine to the subject after fertilization of the egg prevents implantation of the fertilized egg to the uterine walls and therefore, the formation of a fetus and pregnancy is prevented.

By “therapeutically effective” amount, the applicants intend an amount that will inhibit or prevent fertilization in mammals. Such an amount, according to the applicants study, may be in the range of 100 to 200 mg. The most effective amount is about 160 mg of roxatidine.

According to the practice of the invention, the anti-fertility agent, roxatidine may be consumed prior or even after fertilization by any female mammal. The recommended dosage is 200 to 250 mg, per day for a normal adult. Also recommended is continued use of this drug for a period of about 1 to 5 days for better results. Best results are achieved when the anti-fertility agent is administered between the 10^{th} to the 20^{th} day of pregnancy.

A very important aspect of this invention is that roxatidine, as an anti-fertility drug may be consumed before or even after fertilization. Roxatidine has been found to effectively inhibit and/or prevent advancement in pregnancy even after fertilization.

While the focus of the invention is to provide anti-fertility composition employing roxatidine, similar imidazole derivatives (other H₂ Blockers) may be employed since it is merely a matter of routine selection among the different compounds available. Generally, the selection of the anti-fertility agent will be influenced by factors such as efficacy of the compounds in inhibiting decidual cell reaction differential organo-genesis and overall development of the embryo. Accordingly, as mentioned above, several imidazole (H₂ Blockers) derivatives available in the market can be effectively used in the composition. The most appropriate drug selected for purposes of the invention is Roxatidine, although there is no intention to limit the scope of the invention to this drug only.

In accordance with this invention, pharmaceutical compositions containing roxatidine as the primary active ingredient may be prepared. These compositions may be prepared according to any method known to the art for the manufacture of pharmaceutical compositions. Such compositions, if intended for oral use may contain one or more agents selected from the group consisting of sweetening agents, flavoring agents, coloring agents and preserving agents in order to provide pharmaceutically elegant and palatable preparations. Tablets contain the active ingredient roxatidine, in admixture with non-toxic pharmaceutically acceptable excipients that are suitable for the manufacture of tablets. Such excipients may be for example, inert diluents, such as calcium carbonate, sodium carbonate, lactose, calcium phosphate or sodium phosphate; granulating and disintegrating agents, for example, corn starch, or alginic acid, binding agents, for example starch, gelatin or acacia, and lubricating agents, for example magnesium stearate, stearic acid or talc. The tablets may be uncoated or they may be coated by known techniques.

Compositions for oral use may also be presented as hard gelatin capsules wherein the active ingredient roxatidine is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

The compositions may also be formulated as suppositories which can be prepared by mixing roxatidine with suitable nonirritating excipients or carriers such as cocoa butter, polyethyleneglycol or a suppository wax, which are solid at ordinary temperatures but liquid at body temperature and therefore, melt in the rectum or vaginal cavity and release the active component, roxatidine. In other words, all sorts of compositions that are capable of keeping the active ingredient roxatidine, in effective contact with the uterine tissues for sufficient period to bring about deciduoma formation, are envisaged by this invention.

The invention is described in detail by the following examples which should not be construed as limitations on the inventive concept embodied herein.

### EXAMPLE 1

Roxatidine was screened as a post coital anti-fertility agent on different days of pregnancy i.e. day 1^{st}, days 1^{st}-3^{rd} day 4^{th} i.e. day of (estrogen surgery days 1^{st}-5^{th}, days 1^{st}-7^{th} and day 15^{th}-20^{th} i.e. period of organogenesis) post coitum. The aim of the study was to determine the minimum possible dose of drug showing anti implantation activity and the specific days of gestation period when the activity is maximum studies were carried out in order to determine the possible mechanism of action of the drug. Further ELISA was conducted to assess the effects of roxatidine on the serum level of estrogen and progesterone respectively.

Preparation of Drug Solution

Roxatidine acetate is readily and completely soluble in water, thus the solution was prepared by dissolving it in distilled water to obtain a solution of required concentration.

Anti-implantation Studies of Roxatidine in Female Rats

Daily vaginal smears of the female albino rats were taken and the regular periodicity of the estrus cycle was checked.

Procedure for taking Vaginal Smears

The animals were held, ventral side up and a drop of 0.9% saline was inserted into the vagina with the help of a Pasteur pipette, carefully, without damaging the vaginal wall to avoid false positive smears. The drop of normal saline was aspirated and replaced several times with the vaginal fluid, withdrawn and transferred to a microscopic slide. A coverslip was placed carefully on the smear avoiding the air bubble. The slide was then observed under a compound microscope (magnification-45×) and the stage of estrus cycle was determined.

Procedure for Making Permanent Slides

Stain the above slide as follows:

1) Air dry the slide

2) Immerse the slide in absolute methyl alcohol for 5 seconds

3) Dry the slide in air.

4) Apply Geimsa stain (1:50) and keep aside for 30 minutes.

5) Dry the slide and observe it without coverslip under a compound microscope.

Various Stages of the Estrous Cycle in Rats

The estrus cycle is a cascade of hormonal and behavioral events which are highly synchronized and repetitive. The short and precise estrus cycle of the laboratory rat has been a useful model for reproductive studies. The laboratory rat is a spontaneously ovulating, nonseasonal, polyestrus animal. It ovulates every 4-5 days throughout the year unless interrupted by pregnancy or pseudopregnancy.

A century ago, the English scientist Walter Heape described the progressive stages of the estrus cycle. The cycle itself is divided into four stages, centered around the period of estrus when mating behaviour is displayed. He called the period preceding estrus “proestrus, which signifies the period of follicular growth in the ovary, and he termed the period succeeding estrus “metestrus”, a recovery period following ovulation, and “diestrus,” a period when the ovarian secretions from the corpus luteum prepare the uterus for implantation.

The estrus cycle of a rat is usually completed in four to five days. The cycle is roughly divisible into four stages.

1. Proestrous: Proestrous is the beginning of a new cycle. The follicles of the ovary start to mature under the influence of gonadotrophic hormones, and estrogen secretion starts increasing, the smear is characterized by neucleated epithelial cells and this stage lasts for about 12 hours.

2. Estrous: In this stage the uterus is enlarged and extended due to fluid accumulation, estrogen secretion is at its peak. In Estrous stage the smear shows presence squamus cornified cells and is characterized as a period of sexual receptivity, when the female allows copulation. During this stage there is increased running activity. This stage lasts for 12 hours.

3. Metestrous: The ovary contains corpora lutea secreting progesterone. This stage is indicated by the presence of a mixture of nucleated cornified epithelial cells and leukocytes indicating the post ovulatory stage and desquamation of the epithelial cells. Metestrous stage lasts for about 21 hours.

4. Diestrous: the corpora lutea regress and the declining secretion of estrogen and progesterone causes regression of the uterus. The smear shows only leukocytes. This stage is the longest phase of the estrous cycle and has a duration of about 57 hours.

Female rats in proestrus or estrus stage were housed with mature male rats (in the ratio 3 females:1 male). Each female was examined for the presence of spermatozoa in the early morning. The day on which this sign of mating is seen is taken as day 1 of pregnancy. The female was then separated and caged singly. Roxatidine was administered orally to the animal once daily on specific days of pregnancy at different concentrations.

Pregnant rats were divided into 5 groups of 10 animals each. To group I, which served as control, a constant volume of distilled water was administered orally with the help of a catheter. The drug namely, roxatidine was administered at doses of 2.5, 10, 15 & 20 mg/kg body weight orally, on specific days of pregnancy.

### EXAMPLE 2

Anti-implantation Studies in Female Rats using Roxatidiue Acetate when Administered on Day 1 Post Coital

Fifty albino rats were divided into groups of 10 animals each. Group I served as control and received distilled water. Group II to V received Roxatidine acetate at doses of 2.5,10,15 & 20 mg/kg body weight, orally respectively on day 1 postcoital with the help of a catheter.

On day 10 of pregnancy the animal was laprotomized and the number of implants present in both the uterine horns as well as the number of corpora lutea on each ovary was counted. The animals were allowed to complete the gestation period (usually 21-23 days) and the number of litters delivered, if any were counted In this case, 2 litters were delivered.

### EXAMPLE 3

Anti Implantation Studies in Female Rats Using Roxatidine Acetate when Administered on Day 1-3 Post Coital

Fifty albino rats were divided into groups of 10 animals each. Group I served as control and received distilled water. Group II to V received Roxatidine acetate at doses of 2.5,10,15 & 20 mg/kg body weight, orally respectively on day 1-3 postcoital with the help of a catheter.

On day 10 of pregnancy the animal was laprotomized and the number of implants present in both the uterine horns as well as the number of corpora lutea on each ovary was counted. The animals were allowed to complete the gestation period (usually 21-23 days), at the end of which no litters were found.

### EXAMPLE-4

Anti Implantation Studies in Female Rats Using Roxatidine Acetate when Administered on Day 4 Post Coital

Fifty albino rats were divided into groups of 10 animals each. Group I served as control and received distilled water. Group II to V received Roxatidine acetate at doses of 2.5,10,15 & 20 mg/kg body weight, orally respectively on day 4 postcoital with the help of a catheter.

On day 10 of pregnancy the animal was laprotomized and the number of implants present in both the uterine horns as well as the number of corpora lutea on each ovary was counted. The animals were allowed to complete the gestation period (usually 21-23 days) at the end of which 3 litters were found.

### EXAMPLE-5

Anti Implantation Studies in Female Rats Using Roxatidine Acetate when Administered on Day 1-7 Post Coital

Fifty albino rats were divided into groups of 10 animals each. Group I served as control and received distilled water. Group II to V received Roxatidine acetate at doses of 2.5, 10, 15 & 20 mg/kg body weight, orally respectively on day 1-7 postcoital with the help of a catheter.

On day 10 of pregnancy the animal was laprotomized and the number of implants present in both the uterine horns as well as the number of corpora lutea on each ovary was counted. The animals were allowed to complete the gestation period (usually 21-23 days) at the end of which no litters were found.

### EXAMPLE-6

Anti Implantation Studies in Female Rats Using Roxatidine Acetate when Administered on Day 1-5 Post Coital

Fifty albino rats were divided into groups of 10 animals each. Group I served as control and received distilled water. Group II to V received Roxatidine acetate at doses of 2.5,10,15 & 20 mg/kg body weight, orally respectively on day 1-5 postcoital with the help of a catheter.

On day 10 of pregnancy the animal was laprotomized and the number of implants present in both the uterine horns as well as the number of corpora lutea on each ovary was counted. The animals were allowed to complete the gestation period (usually 21-23 days) at the end of which no litters were found.

### EXAMPLE-7

Anti Implantation Studies in Female Rats Using Roxatidine Acetate when Administered on Day 15-20 Post Coital

Fifty albino rats were divided into groups of 10 animals each. Group I served as control and received distilled water. Group II to V received Roxatidine acetate at doses of 2.5,10,15 & 20 mg/kg body weight, orally respectively on day 15-20 postcoital with the help of a catheter.

On day 10 of pregnancy the animal was laprotomized and the number of implants present in both the uterine horns as well as the number of corpora lutea on each ovary was counted. The animals were allowed to complete the gestation period (usually 21-23 days) at the end of which 2 litters were seen.

### EXAMPLE-8

Anti-ovulatory Effects of Roxatidine Acetate in Immature Female Rabbits Using Copper Acetate Induced Ovulation Method

Immature female rabbits of 800 gms-1 kg were divided into six groups of three animals each. Group I served as first control and received copper acetate only. Group II served as second control and received distilled water only (vehicle for roxatidine acetate). Group III, IV, V & IV received roxatidine acetate in the doses of 2.5, 10, 15 & 20 mg/g body weight, respectively.

### EXAMPLE-9

Preovulatory Effects of Roxatidine Acetate in Female Albino Rats

Animals with normal estrus cycle were divided into 4 groups with 10 animals in each group. Group I served as control and received distilled water orally. Animals in Group II, II, IV & V were administered roxatidine acetate at the doses of 2.5, 10, 15, & 20 mg/kg body weight respectively, for 15 consecutive days. During this period daily vaginal smears were taken to observe the regularity of the estrus cycle.

The females in all the groups were then paired with males of proven fertility. Daily vaginal smears were taken until the animals successfully mated. This was indicated by the presence of spermatozoa in the vaginal smears and this was taken as day 1 of pregnancy. On day 10 each animal was laprotomized and the no. of implants/resorption sites and the no. of corpora lutea were counted. The number of litters delivered at the end of the gestation period were 2.

1. A method for controlling fertility in mammals, said method comprising the step of administering a therapeutically effective amount of roxatidine to the mammals.

2. A method as claimed in claim 1 wherein, the effective amount of roxatidine is in the range of 100 to 300 mg per day.

3. A method as claimed in claim 1 wherein, the dosage of administration of roxatidine is 200 to 250 mg per day.

4. A method as claimed in claim 1 wherein, the preferred amount of roxatidine is 160 mg per day.

5. A method as claimed in claim 1 wherein, roxatidine as an anti-fertility agent is administered to a mammal between the 10^{th} to 20^{th} day of pregnancy.

6. A method as claimed in claim 1 wherein, the period of administration of roxatidine is 1 to 5 days.

7. A method as claimed in claim 1 wherein, the routes of administration are selected from oral, parental and anal.

8. A method as claimed in claim 1 wherein, roxatidine is formulated in physical forms selected from tablets, capsules, syrups and injectibles for administration.

9. A method for inducing abortion or preventing the implantation of the fertilized egg to the uterine wall in mammals including humans, said method comprising the step of administering a pharmaceutically effective amount of a substance essentially comprising roxatidine to the mammal.

10. A method as claimed in claim 9 wherein, the substance comprises pharmaceutically acceptable carriers and additives.

11. A method as claimed in claim 9 wherein, the substance containing roxatidine is formulated in physical forms selected from tablets, capsules, syrups and injectibles for administration.

12. Use of roxatidine as an anti-fertility agent or to induce abortion or prevent implantation of the fertilized egg to the uterine wall in mammals by administering a therapeutically effective amount of roxatadine to the mammal.

13. Use of roxatidine for the manufacture of a composition capable of being used as an anti-fertility agent.

14. Use as claimed in claim 12 or 13 wherein, the amount of roxatidine in the composition is 100 to 300 mg per day.

15. Use as claimed in claim 12 or 13 wherein the dosage of administration of roxatidine is 200 to 250 mg per day.

16. Use as claimed in claim 12 or 13 wherein, the preferred amount of roxatidine in the composition is 160 mg per day.

17. Use as claimed in claim 12 or 13 wherein roxatidine as an anti-fertility agent is administered to a mammal between the 10^{th} to 20^{th} day of pregnancy.

18. Use as claimed in claim 12 or 13 wherein the period of administration of roxatidine is 1 to 5 days.
